(19) United States
(12) Patent Application Publication
(10) Pub. No.: US 2018/0350455 A1
(43) Pub. Date: Dec. 6, 2018

(54) SENSOR-ENABLED MOBILE HEALTH MONITORING AND DIAGNOSIS

(71) Applicant: LifeWIRE Corporation, Toronto (CA)

(72) Inventor: Howard ROSEN, Toronto (CA)

(73) Assignee: LifeWIRE Corp.

(21) Appl. No.: 15/989,558

(22) Filed: May 25, 2018

Related U.S. Application Data
(60) Provisional application No. 62/512,639, filed on May 30, 2017.

(51) Int. Cl. G16H 10/60; G16H 80/00; G06F 17/30

(52) U.S. Cl. CPC G16H 10/60; G06F 17/30864; G16H 80/00

(57) ABSTRACT

A computerized system, method, and computer program product is provided for predictive detection of health conditions using sensor-based quantitative data, and subjective qualitative data. Embodiments provide sending messages to a first device associated with a patient according to a query setting, receiving first patient data comprising responses to the messages, receiving second patient data comprising sensor data corresponding to the patient from a second device associated with the patient, performing computational analysis on the first patient data and the second patient data to generate third patient data, and generating a unique digital fingerprint for the patient based on the first patient data, the second patient data, and the third patient data.

### CROSS-REFERENCE TO RELATED APPLICATIONS

This application claims the benefit of U.S. Provisional Application No. 62/512,639, filed on May 30, 2017, which is incorporated herein by reference in its entirety.

### TECHNICAL FIELD

This disclosure generally relates to communication systems for monitoring and diagnosing physiologic functions of a user, including health conditions. More particularly, this disclosure relates to computerized interactive communication systems for monitoring and diagnosing a user's health, including the use of subjective data received from a user, objective data received from at least one sensor associated with the user, and combinations and data derived therefrom.

### BACKGROUND

For patients suffering from chronic illness, monitoring of their condition and ensuring compliance of health regimes is vital to their long term health. However, patients are often unsuccessful at keeping proper records of health statistics related to their condition. Logbooks, PDAs and computers adapted to monitor their conditions often become a burden, furthering their suffering instead of being a tool to assist them in effectively dealing with their condition.

In general, the ability to ensure the monitoring and response to changing physiological conditions in real time gives the patient or a caregiver the ability to react to their condition before an emergency occurs. The improved care through direct patient management means reduced costs associated with chronic conditions: less physician visits, less hospital visits, and fewer sick days.

For example, 40% of doctor visits and hospital re-admissions result from failure to take prescription medicine properly and on time. It is thus important for health care providers and clinicians to improve and ensure patient adherence to prescription routines. Ensuring patient adherence to other clinician-based guidance is also important in improving post-operative care, maternal care, and general wellness. In addition, patients suffering from behavioral issues such as high risk suicide, Post-Traumatic Stress Disorder (PTSD), and substance abuse can also benefit from outreach programs and patient monitoring.

While constant communication with and monitoring of patients substantially improves patient healthcare management, such efforts are challenging for clinicians and healthcare providers to effectively undertake. For example, current communication and monitoring methods require clinicians to gather information from disparate systems and devices, and manually contact patients to gather information for proper monitoring and diagnoses.

In view of the foregoing, there is a need for improved systems and methods for automating a continuous communications protocol with patients for improved patient monitoring and healthcare management. One of ordinary skill will understand from this disclosure that other uses for the presented embodiments are possible as well.

### SUMMARY

Embodiments of the present disclosure include sensor-enabled computerized systems and methods for monitoring and diagnosing patient health through a controlled feedback and notification system. Other embodiments and features are also presented in this disclosure.

In some embodiments, a computerized system may monitor both quantitative and qualitative patient information responses and data, automating a continuous two-way, three-dimensional dialogue and outreach between the system and a user, such as a patient. The interactive exchanges in combination with sensor data collected from a sensor device such as a wearable device, and messaging and transactional analytics data, may be tracked against advanced algorithms that gain insight into a probable condition of the user, thereby generating one or more notifications so that providers can be pre-emptive. In this manner, the disclosed embodiments can provide a marked improvement over the subjective, manual, and multi-step processes known in the prior art.

In some embodiments, a computer system may combine collected subjective data via patient responses through a personal communication device, with objective data collected from a sensor device or sensor of the communication device, to generate combined and correlated data for the user. In some embodiments, the system may generate new data based on the combination of subjective and objective data, and/or derive data from the collected data or combinations thereof. Collected, generated, and derived data may be used to generate and update a unique digital ‘fingerprint” of that user.

In one embodiment, a computerized system for monitoring patient health is provided. The system includes a storage device that stores a set of instructions and at least one processor configured to execute the stored instructions to perform operations comprising sending, to a first device associated with a patient, messages according to a query setting, and receiving, from the first device associated with the patient, first patient data comprising responses to the messages. The operations further comprise receiving, from a second device associated with the patient, second patient data comprising sensor data corresponding to the patient. The operations further comprise performing computational analysis on the first patient data and the second patient data to generate transactional analytic data, and generating a unique digital fingerprint for the patient based on the first patient data, the second patient data, and the transactional analytic data. The unique digital fingerprint may be stored in a database and used to update treatment plans for the patient.

In one embodiment, a computer-implemented method for monitoring patient health is provided. The method is performed by at least one processor and comprises sending, to a first device associated with a patient, messages according to a query setting, and receiving, from the first device associated with the patient, first patient data comprising responses to the messages. The method further comprises receiving, from a second device associated with the patient, second patient data comprising sensor data corresponding to the patient. The method further comprises performing computational analysis on the first patient data and the second patient data to generate transactional analytic data, and generating a unique digital fingerprint for the patient based on the first patient data, the second patient data, and the transactional analytic data.

In one embodiment, a tangible, non-transitory computer-readable memory device is provided. The memory device stores a set of instructions that, when executed by at least one processor, cause the at least one processor to perform operations comprising sending, to a first device associated with a patient, messages according to a query setting, and receiving, from the first device associated with the patient, first patient data comprising responses to the messages. The operations further comprise receiving, from a second device associated with the patient, second patient data comprising sensor data corresponding to the patient. The operations further comprise performing computational analysis on the first patient data and the second patient data to generate transactional analytic data, and generating a unique digital fingerprint for the patient based on the first patient data, the second patient data, and the transactional analytic data.

In some embodiments, the unique digital fingerprint comprises at least one of a predictive model or behavioral patterns for the patient.

In some embodiments, the system provides for feedback and measurement for healthcare providers to ensure that those under their care are complying with notifications and respective regimes.

In some embodiments, the system and method provides a simple, cost effective, and flexible patient feedback and management system. The system provides for a long term management, compliance and analysis for the benefit of the individual. In implementing this method in a healthcare environment, the individual will gain a better understanding of managing their lifestyle and behavior and allow for healthcare managers to measure compliance of certain activities (if required). Further, employing already existing and available low cost technology improves the rate of patient data capture.

In some embodiments, the system may facilitate self-management of conditions by the patients. Users may be responsible for tailoring the setup to their particular needs. Queries, notifications, reminders, and/or alerts are provided on a regular basis, prompting users to take necessary quantitative or qualitative measurements, medication, or to record particular activities or conditions. These notifications can be tailored in a number of ways. For example, the users can choose to monitor the particular attribute or attributes that are relevant to their condition, including glucose level, temperature, blood pressure, heart rate, weight, medication intake, pain characteristics, etc. The user is able to specify the number of notifications, and schedule them appropriately. The system confirms the input from the user, resulting in a reduction in the possibility of error. Such input in monitored, allowing the ability to ensure compliance or to determine the extent of compliance of the user.

The system comprises two separate interfaces in this respect: (i) a consumer interface; and (ii) a system administrator interface. The user is prompted for information, e.g., notification attributes, and the information is submitted back to the server. Based on disease-specific protocols determined by the user, the data can be analyzed immediately to identity trends, especially the detection of worsening conditions. Based on user-configured rules, family and other care-givers can be notified immediately of any adverse trends.

Users and their healthcare network can access more detailed information regarding the data collected through a web interface. The raw input data, basic trends and charts show the patients' most recent information as well as historical information which can all be printed out. Analytical tools can also be created and linked with the database. In addition, the healthcare provider and/or caregiver can also, based on the information, ‘push’ key messages via the disclosed system to a user or group of users depending on the circumstances.

The disclosed system also contemplates the pairing of other hardware, apart from personal communication devices, to receive relevant patient data for analysis. For example, patients can link sensor devices, wearable devices, “smart” wireless enabled glucometers, or similar ‘smart’ monitors that include various sensors directly with the system or with their personal communication devices. Furthermore, a global positioning device could be linked with the system or within the communication device to provide patient location monitoring or analytics as to past or future location-related behavior. Hardware can also be adapted within the communication device such as biometric sensors to allow for quantitative measurements of the various attributes.

Further, the disclosed system is also operable to prompt the user for specific non-quantitative health or lifestyle related information related to any of the applications discussed herein.

In further aspects of the disclosed embodiments, the monitoring system can be utilized for a variety of purposes, including both health and non-health related purposes. For example, the system may be used for monitoring patients to reduce or prevent suicide ideation and/or attempts; monitoring patients suffering from substance abuse, PTSD, and/or depression; monitoring first responders; monitoring military personnel; maintaining contact with children; maintaining conduct with adults requiring supervision, such as with prison inmates, people involved in extreme-style sports such as skiing or cross country running, walkers, joggers, bike riders, etc.; meeting notification; reminder notification; travel or location updates; any type of location/trend analysis, including trend analysis involving a combination of location/activity and other data (e.g., tracking activity using blood pressure); general remote input of an activity on a regular basis and analyzed for access by user and approved user(s); and/or general notification reminder system based on pre-determined parameters.

### BRIEF DESCRIPTION OF THE DRAWINGS

The accompanying drawings, which are incorporated in and constitute a part of this disclosure, illustrate several embodiments and aspects of the present disclosure, and together with the description, serve to explain certain principles of the presently disclosed embodiments.

FIG. 1 illustrates an exemplary system environment for implementing embodiments of the present disclosure.

FIG. 2 illustrates an exemplary graphical user interface (GUI) for a device associated with a first user, according to some embodiments of the present disclosure.

FIG. 3 illustrates an exemplary interface for the system of FIG. 1, according to some embodiments of the present disclosure.

FIG. 4 illustrates an exemplary GUI for a device associated with a second user, according to some embodiments of the present disclosure.

FIG. 5 illustrates an exemplary device for collecting and transmitting sensor data, according to some embodiments of the present disclosure.

FIG. 6A illustrates a flow diagram of an exemplary process performed by the system, according to some embodiments of the present disclosure.

FIG. 6B illustrates a flow diagram of an exemplary process for setting up user information, according to some embodiments of the present disclosure.

FIG. 6C illustrates a flow diagram of an exemplary approval process, according to some embodiments of the present disclosure.

FIG. 7 illustrates a flowchart of an exemplary process for monitoring patient health, according to some embodiments of the present disclosure.

FIG. 8 illustrates an exemplary clinical interface, according to some embodiments of the present disclosure.

FIGS. 9A-B illustrate exemplary server systems, according to some embodiments of the present disclosure.

FIG. 10 illustrates a flow diagram of an exemplary process for the system workflow, according to some embodiments of the present disclosure.

FIG. 11 illustrates a flow diagram of an exemplary process for protecting the information, according to some embodiments of the present disclosure.

FIG. 12A illustrates a flowchart of an exemplary process for a system administrator interface, according to some embodiments of the present disclosure.

FIG. 12B illustrates a flowchart of an exemplary process for a system administrator compliance review, according to some embodiments of the present disclosure.

In the drawings, embodiments of the disclosure is illustrated by way of example. It is to be expressly understood that the description and drawings are only for the purpose of illustration and as an aid to understanding, and are not intended as a definition of the limits of the disclosure or of any particular embodiment discussed herein.

### DETAILED DESCRIPTION

Some embodiments of the present disclosure will now be described. One of ordinary skill will understand that variations from these embodiments is possible.

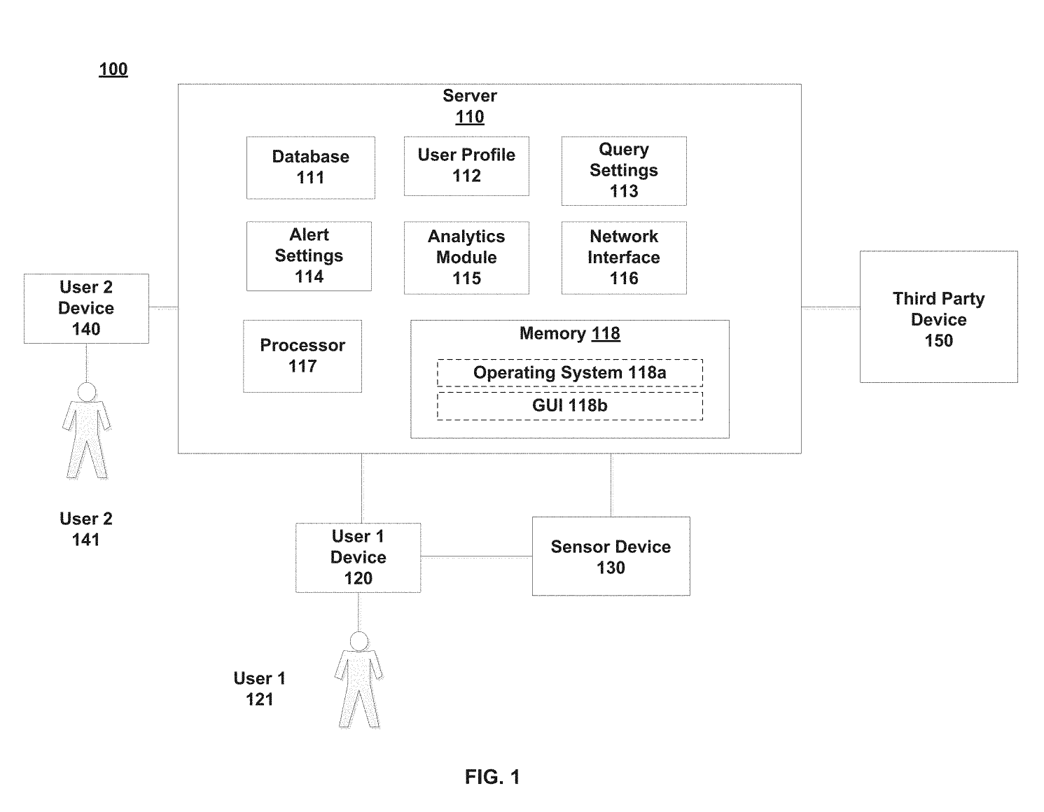

FIG. 1 illustrates an exemplary system environment 100 for implementing embodiments of the present disclosure. As shown in FIG. 1, system environment 100 may include a server 110, a device 120 for a first user 121, a sensor device 130 for first user 121, a device 140 for second user 141, and a third party device 150. Each of these devices may be implemented as hardware, software, firmware, or a combination thereof. The number and arrangement of components in FIG. 1 is presented for purposes of illustration. Additions, modifications, and substitutions can be made to these components, as needed. Signals or data may be sent between these devices.

Server 110 may comprise a database 111, a user profile 112, query settings 113, alert settings 114, analytics module 115, network interface 116, processor 117, and memory 118. Memory 118 may store instructions or software code for causing processor 117 to execute operating system 118a and provide for display a GUI 118b. Each of these modules may be implemented as hardware, software, firmware, or a combination thereof. Signals or data may be sent between these modules.

Database 111 may include one or more computing devices configured to may receive and store data associated with one or more processes disclosed herein, under the control of server 110 and/or one or more other processors in communication with database 111. For example, database 111 may store one or more programs or other computer-executable code that, when executed, cause one or more processors to perform functions and methods associated with disclosed embodiments. Database 111 may also be implemented as storage for other data. For example, database 111 may store data for use by the user profile 112, query settings, 113, alert settings 114, and/or analytics module 115. In some embodiments, database 111 is located remotely from server 110.

User profile 112 may include user information and any information that may be needed to set up a user account and access system 100. For example, user profile 112 may include information such as an account username, account password, contact information, preferences, personal information, and a user tag. User profile 112 may also include health data relating to the user's medical records.

Query settings 113 may include settings for messaging and monitoring parameters. The query settings may include, for example, a query schedule. The query schedule may determine a schedule and/or frequency that one or more queries are sent to the user's device. The query schedule, for example, may include information for determining the timing and frequency for sending queries to a user device. In some embodiments, the query schedule may include the number of queries the user receives in one day. In some examples, the user may elect to receive a range of queries, such as one to six queries per day. In other examples, the user may elect to receive more than six queries per day. In yet another example, the user may select not to receive any query. Additionally, the query schedule may include a specific time and/or date at which one or more queries should be sent to the user's device, or a specific time and/or date at which one or more queries should not be sent to the user's device.

In some embodiments, the query schedule may include query templates and query procedures for providing communications with users and/or third parties within system 100. The query schedule may also include information relating to the users and user devices the queries are sent to. The query settings may further include a query type. The query type may be, for example, one of: a single query, multiple queries with no immediate response required, and multiple queries with a response dependent on input.

Alert settings 114 may include alert settings for sending alerts and notifications. The alert settings may include at least an alert criterion and an alert procedure that corresponds to each alert criterion. The alert criterion may define a condition in which the alert procedure is executed. For example, the alert criterion may be defined such that the alert procedure is executed when a response data to a query is above or below a predetermined value. In another example, the alert criterion may be defined such that the alert procedure is executed when a response data for a query is above or below the cumulative average of the response data.

In some embodiments, the alert procedure may define a set of actions executed by the system when the alert criterion is met. The set of actions may include contacting one or more people based on information the user entered while configuring settings for notification and monitoring parameters. For example, when an alert procedure is executed, system 100 may retrieve a stored list of individuals identified in the settings for notification and monitoring parameters and contact everyone in the list using a messaging protocol such as an SMS message. The SMS message may include a message indicating that the user is in need of assistance, for example. The alert settings may also include preferences relating to the alerts, including the type of alert, volume settings, notification settings, etc.

Analytics module 115 may include one or more computing components configured to analyze user data to determine patterns, changes in patterns, predictive modeling, and dissonance between the data. User data may include any data relating to the user, including data retrieved from database 111, user profile 112, responses to queries received from user devices 120 and 140, sensor and other data retrieved from device 130, and/or data retrieved from third party 150. In some embodiments, analytics module 115 may compare the responses to the queries to previously determined patterns and/or responses from the user, such as pattern or historical response data stored in a digital fingerprint for the user. In some embodiments, analytics module 115 may use time stamp information to determine the difference in time between a sent query and a response from the user. Such time differential data may be merged with other data sets and analytics in analytics module 115 to generate a unique digital fingerprint for the user. In some embodiments, analytics module 115 may analyze the user data to determine a further query or set of queries, from database 111 and/or query settings 113, to send to user devices 120 and 140. In some embodiments, the user data includes patient information and physiological data. In some embodiments, analytics module 115 may be implemented on device 120 or 140.

Device 120 of user 121 and device 140 of user 141 represent client devices utilized by users. Examples of such devices include, for example, a mobile device, a tablet, a laptop, a cellular device, a personal computer, a smartphone (e.g., Apple iPhone, Blackberry, Android-based phones, etc.), wearable smart device such as a smart watch, notebooks, etc. Devices 120 and 140 may include a network component to communicate with server 110 via network interface 116 over a communications network. The network interface 116 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, Bluetooth™, infrared, or long or short range wireless transmission protocols. The communication network may include, without limitation, a direct interconnection, local area network (LAN), wide area network (WAN), wireless network (e.g., using Wireless Application Protocol), the Internet, a cellular network, or any other type of network.

In some embodiments, devices 120 and 140 communicate with server 110 using Short Message Service (“SMS”) and Multimedia Message Service (“MMS”) technology. In addition, a skilled artisan would recognize that the disclosed system may utilize alternative technology such as email, social-media messages/notifications, smartphone App notifications, or smartwatch notifications.

Devices 120 and 140 may further include an input component, such as a number pad, keyboard, touch screen, voice recognition system, or other input mechanism. Devices 120 and 140 may also include a display for displaying a graphical user interface (GUI) to the user. Devices 120 and 140 may further include sensors for collecting biometric or physiological information, and location-determination components such as a Global Positioning Sensor (GPS). Devices 120 and 140 may include one or more processors configured to execute software instructions stored in memory, such as memory included in devices 120 and 140. Devices 120 and 140 may include software that when executed by a processor performs Internet-related communication and content display processes. For instance, the devices may execute browser software that generates and displays interfaces including content on a display device included in, or connected to, devices 120 and 140. Devices 120 and 140 may execute applications that allow communication with components of system 100. The disclosed embodiments are not limited to any particular configuration of devices 120 and 140. For instance, devices 120 and 140 can be devices that store and execute mobile applications that interact with server 110 and/or sensor device 130 to perform aspects of the disclosed embodiments.

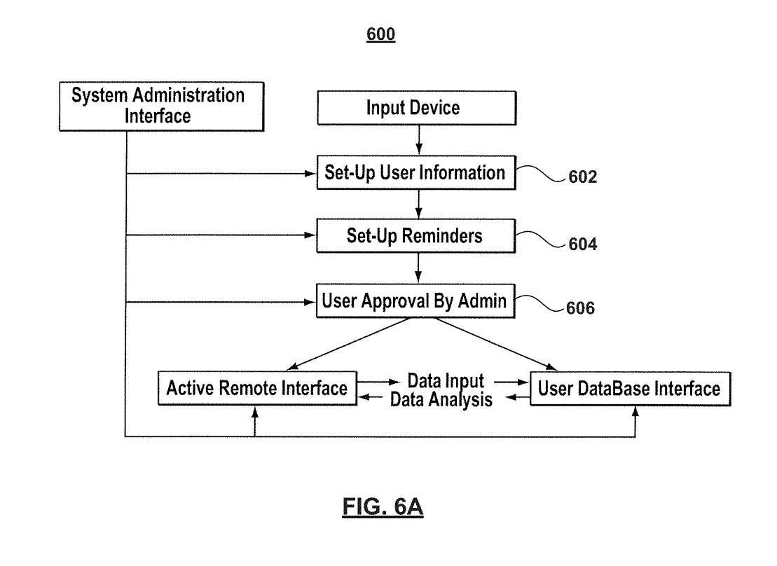

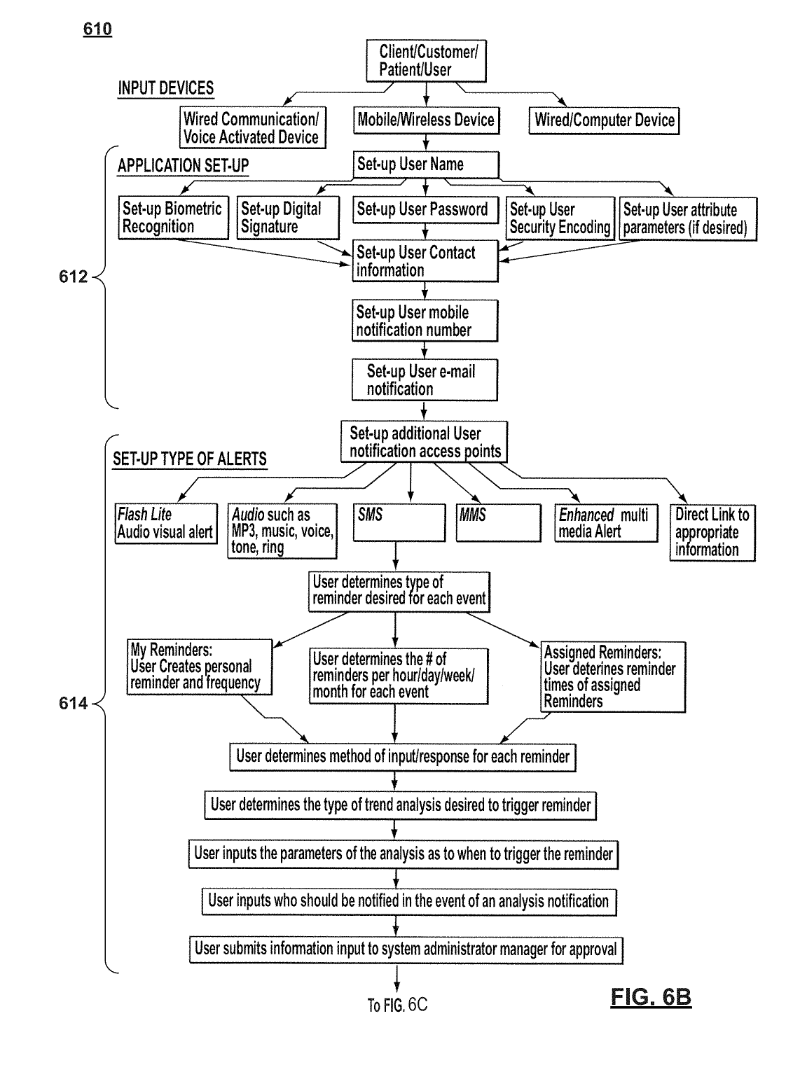

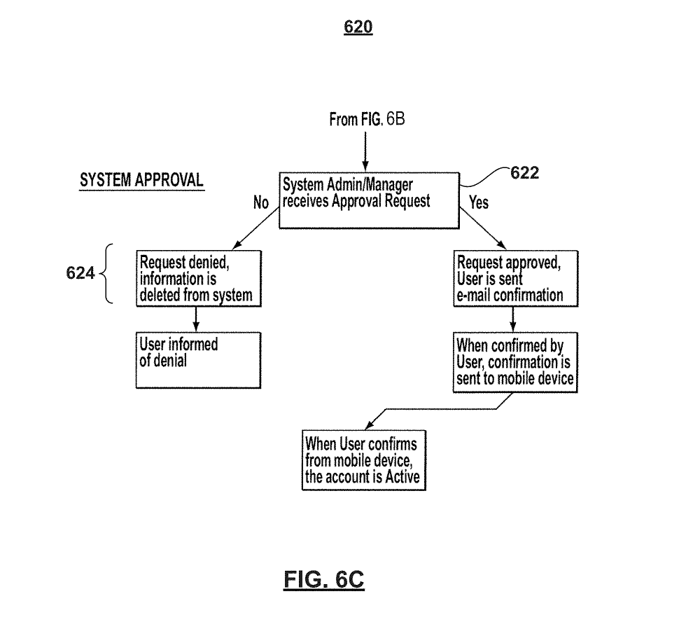

In some embodiments, user 121 may be a patient utilizing the services provided by server 110. To access the system, each patient may be required to complete a registration system, as further disclosed in FIGS. 6A-C below. During registration, user 121 may provide personal information that is stored in a data structure for a user profile 112.

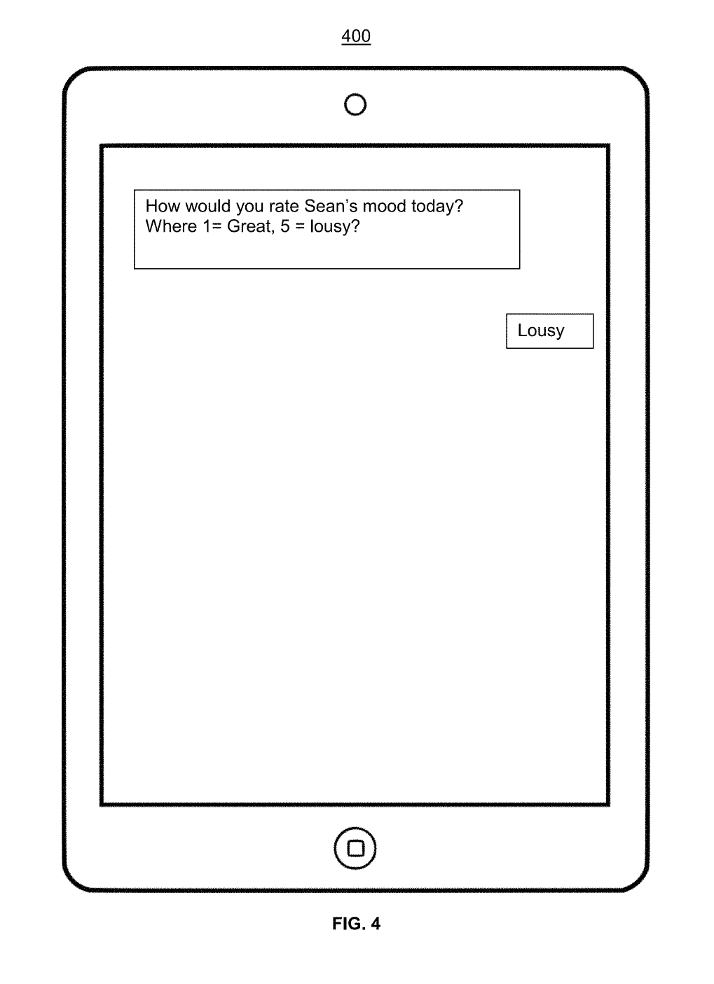

In some embodiments, user 121 provides contact information for further users. For example, user 121 may provide contact information for contacting a user device 140 of a second user 141. Second user 141 may be a spouse, family member, friend, or care giver for user 121. As disclosed below with respect to FIG. 4, server 110 may send messages to user 141 to solicit responses to queries relating to user 121. The responses from user 141 may be stored in database 111. The responses from user 141 may further be processed by analytics module 115 for generating the unique digital fingerprint for user 121.

Sensor device 130 may be associated with user 121 and may include one or more sensors for measuring one or more parameters relating to user 121. For example, sensor device 130 may include sensors for measuring physiological or biometric data from user 121. Sensor device 130 may communicate sensor data from the one or more sensors to device 120 and/or directly to server 110. In some embodiments, sensor device 130 may be a wearable device and may include a wearable component such as a band or clip that enables the wearable device to be attached to the body of user 121. For example, the wearable device may be worn around a user's wrist, finger, waist, ankle, foot, or other body part. The wearable device may also be attached to the user's chest, torso, or other body party to collect the sensor data. In some embodiments, the sensor device 130 is an implantable device with implantable sensors. In some embodiments, sensor device 130 may include a personal digital assistant (for example, Apple® Siri, Microsoft® Cortana, OK Google™, Amazon™ Alexa, etc.). In some embodiments, sensor device 130 may be integrated into device 120.

Third party device 150 may be a device for any third party authorized to receive notifications, monitoring data, and/or alerts regarding user 121. For example, third party device may be, for example, a mobile device, a tablet, a laptop, a cellular device, a personal computer, or the like. The third party may be, for example, a hospital, doctor, psychologist, counselor, friend, family member, clinician, or the like. Server 110 may communicate with third party device 150 over a communications network, such as the Internet, a local area network, a wide area network, a cellular network, a wireless network, or any other type of network.

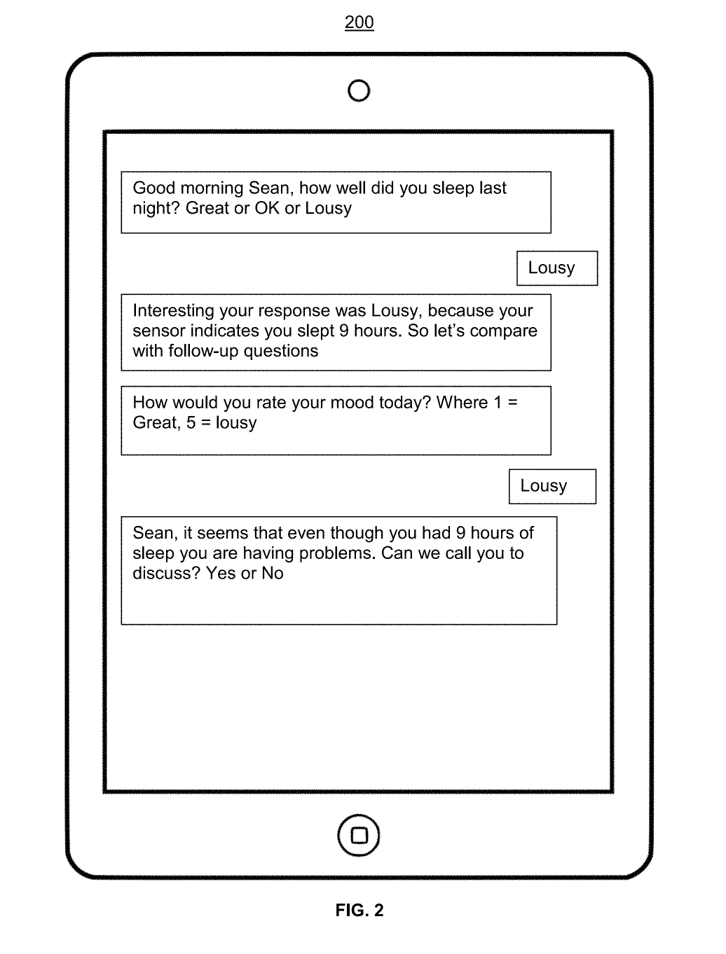

FIG. 2 illustrates an example graphical user interface (GUI) 200 for device 120 associated with user 121. For example, GUI 200 may include a first interface for an application executed by the user device 120. As depicted in GUI 200, a user may receive personalized messages from server 110. The messages may be generated and transmitted using data stored on database 111 and/or based on parameters stored in query settings 113. For example, server 110 may send a message to user device 120 that solicits a response from user 121. In some embodiments, the messages may solicit subjective qualitative and/or quantitative data relating to the user's mood, behavior, and mental or physical state. As one example, the server 110 may send a message asking user 121 to rate their sleep in the previous night by responding to the message using a rating scale. For example, the user may be asked to rate their sleep as either “great,” “OK,” or “lousy.”

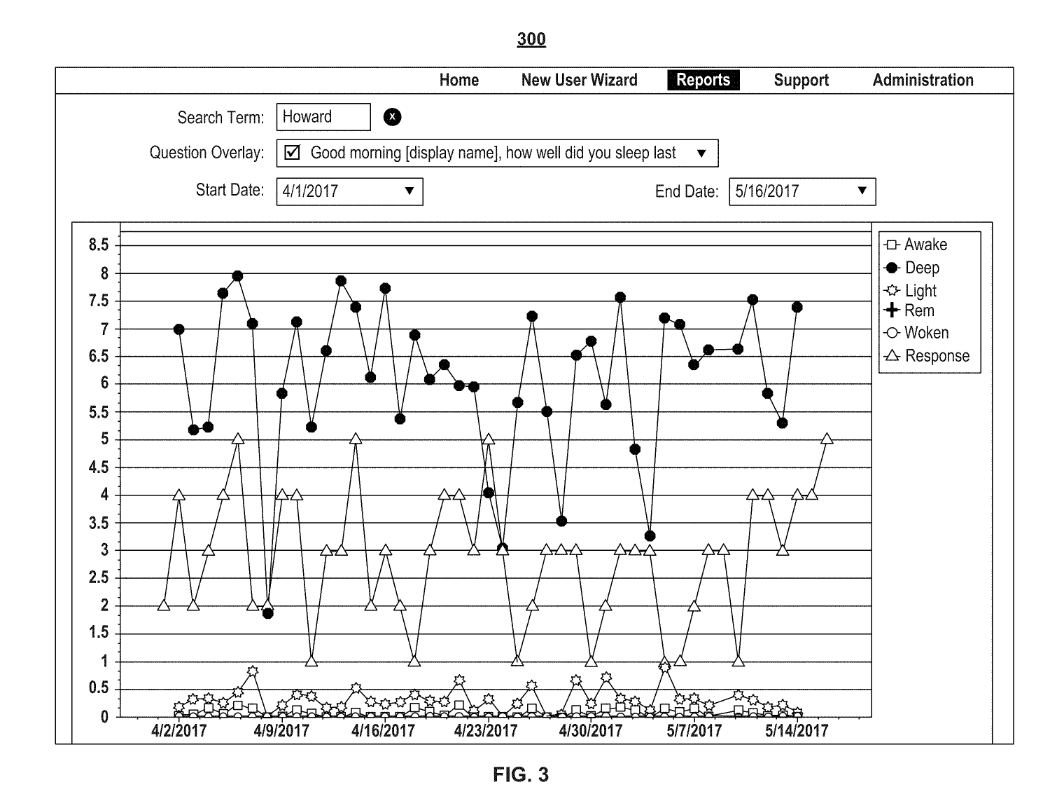

As shown in FIG. 2, once server 110 receives the user's response, the system may compare the response to data stored in user profile 112 or database 111. The comparison may yield a determination that the user's response does not correlate with the stored data. In some embodiments, the stored data may include sensor data received from sensor device 130. For example, if user 121 rates his sleep as “Lousy,” server 110 may compare this answer to sleep data received from one or more sensors in sensor device 130. As shown in FIG. 3, sleep data for a user received from the sensor device 130 may be analyzed. For example, the sleep data for the previous night may be compared to the user's rating to determine any deviations or correlations. The comparison may be implemented by analytics module 115.

Based on the comparison, server 110 may determine that further follow-up questions are warranted. For example, a follow-up question may request user 121 to rate their mood on a scale of 1 to 5, where 1 is great and 5 is lousy. The follow-up question may be determined based on the query settings, for example. Here, the user's response to the follow-up question may further be compared to data stored in user profile 112 or database 111.

In some embodiments, server 110 retrieves contact information for a device 140 of a designated second user 141. Second user 141 may be a family member, spouse, caregiver, or other designated person with first-hand information of user 121. Based on the responses from user 121, server 110 may send one or more messages, similar to the messages sent to user 121, soliciting feedback responses from user 141. For example, FIG. 4 illustrates an example graphical user interface (GUI) 400 for device 140 associated with user 141. For example, GUI 400 may include a second interface for an application executed by the user device 140. As depicted in GUI 400, a second user may receive personalized messages from server 110. The messages may be generated and transmitted using data stored on database 111 and/or based on parameters stored in query settings 113.

Server 110 may send a message to user device 140 that solicits a response relating to user 121. For example, a message may be sent to device 140 requesting user 141 to rate the mood of user 121 on a scale of 1 to 5, where 1 is great and 5 is lousy. Analytics module 115 may compare the response from the second user 141 to the response from user 121 to the same message (as depicted in FIG. 2). Analytics module 115 may analyze the responses from users 121 and 141, and determine a correlation, discrepancy or difference in opinion. Based on the determination, server 110 may send a follow-up message to user device 120 requesting further action. In some embodiments, the follow-up message may request a phone call, meeting, or other communication between user 121 and a third party, such as a doctor, psychologist, counselor, friend, family member, clinician, or the like.

Referring to FIG. 3 once again, the graph may display data relating to the different types of sleep a user may have. For example, as shown in the graph, a user's sleep may be generally categorized as awake, deep, light, rem, woken, and response. Detectable types of sleep may vary based on the sensing and processing capabilities of sensor device 130. Each type of sleep includes a set of data points received from sensor device 130. The set of data points for each type of sleep may extend across a time period and create a base line and further data points to analyze. For example, data points for REM sleep may be analyzed and compared to the data points for light sleep to determine how each may have affected the user. In some embodiments, the analysis may cause further sets of queries to be sent to the user.

In some embodiments, the dashboard displayed in FIG. 3 includes a number of selectable options for selecting specific message queries for comparison to sensor data. The selectable options may be selected by an administrator or by server 110. In this way, qualitative data may be compared graphically and may trigger further analysis. For example, the analysis may trigger further queries to send to the user, pre-existing protocols retrieved from database 111 or query settings 113, further analysis by analytics module 115, and/or notifications or alerts by alert settings 114. In some embodiments, analytics module 115 may analyze all data points and/or responses to queries to trigger further queries to send to the user, pre-existing protocols retrieved from database 111 or query settings 113, further analysis by analytics module 115, and/or notifications or alerts by alert settings 114.

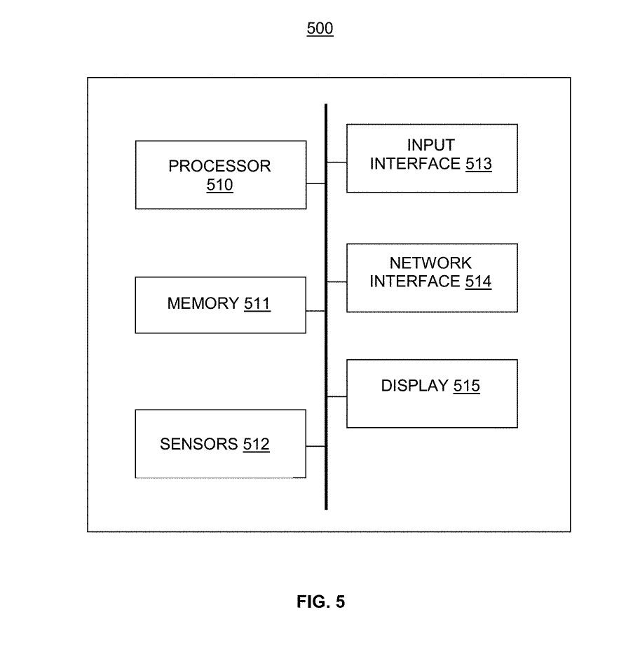

FIG. 5 illustrates an exemplary device 500 for collecting and transmitting sensor data. In some embodiments, device 500 is a wearable device, an implantable medical device, or a personal digital assistant device. Device 500 may comprise a processor 510, a memory 511, one or more sensors 512, input/output interface 513, network interface 514, and display 515. Each of these modules may be implemented as hardware, software, firmware, or a combination thereof. Signals or data may be sent between these modules. In addition, each of these modules may be mounted or enclosed within a central housing and attached to a wearable component. Sensors 512 may gather physiological, biometric data and/or physical data. For example, sensors 512 may gather data relating to heart rate, pulse rate, electrocardiography (EKG or ECG), respiration rate, body temperature, skin temperature, blood pressure, glucose levels, activity levels, body position or orientation, location, sound, or sleep activity. In some embodiments, sensors 512 are implantable in the body of a user.

Sensor data collected from sensors 512 may be stored in memory 511 and/or transmitted over network interface 514 to other devices, such as devices 120, 130, 140, or server 110. The network interface 514 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, Bluetooth™, infrared, wired communication protocols, or other short range or long range wireless transmission protocols. In some embodiments, device 500 includes an input interface 513 such as a keypad, button, keyboard, or other input device. Device 500 may also include an optional display 515 for displaying information, such as sensor data, alerts, connectivity, or the like. In some embodiments, device 500 is integrated into device 120 such that device 120 includes sensors 512.

FIG. 6A is a flow diagram of an exemplary process 600, consistent with disclosed embodiments. Process 600 may be performed by a system such as system 100.

At step 602, the user may set-up a user account using an input component of a device, such as device 120. In systems that use a voice-recognition system, the information entered by the user may be verified for accuracy in an additional step. In one example, a user may setup an account using device 120 configured to communicate with the server 110 via a communications network such as the Internet. For example, the user may use device 120 to access a system website hosted on server 110 or a web server associated with server 110, and enter a set of information requested by the system website using input devices of device 120. The set of information requested may include user information and other information that may be needed to set up a user account and stored as user profile 112. In some embodiments, the information may include information to password protect the user account. In some embodiments, the user may be a patient or a person acting on behalf of the patient.

In some embodiments, at least one custom-defined attribute (“user tag”) may be created and associated with the user account. In some embodiments, each user tag may include a key and a value. The key of the user tag may include a description of a data, and the value of the user tag may include the data itself. The user tag may be associated with an item to be included in a query to or from the user. For example, a key of the user tag may be the text “birthday” and the value of the user tag may be the birthday of the user. In another example, a key of the user tag may be the text “delivery date,” and a value of the user tag may be the delivery date of the pregnant user. In yet another example, a key of the user tag may be the text “nickname” and the value of the user tag may be the preferred nickname of the patient. In a further example, a key of the user tag may be the text “doctor” and the value of the user tag may be a name or identification of a physician associated with the user. In some embodiments, one or more user tags may be created and associated with a user account by the user. In these embodiments, the user may enter the key and the value of the user tags using while creating the user tag. In other embodiments, the user may select a predetermined key, and enter a value of the user tag.

Alternatively, or additionally, one or more user tags may be created and associated with one or more user accounts by an administrator. In some examples, the administrator may enter both the key and the value (i.e., default value) for each of the user tags created by the administrator, and the user may change the values of one or more user tags created by the administrator at a later time. Alternatively, the administrator may enter only the key for each of the user tags created by the administrator, and the user may be required to enter the initial values of the user tags created by the administrator during the user account set-up process. In some embodiments, the administrator may be one of healthcare professionals caring for the patient. In some embodiments, the administrator may load a set of user tags that was pre-created and associate the loaded user tags with one or more user accounts.

In some embodiments, a value of a user tag may be generated automatically by the system. For example, a key of a user tag may be the text “Capitals Fan” and a value of the user tag may be the latest hockey game score involving the Capitals team retrieved by server 110 of the system from the Internet. In some embodiments, a value of a user tag may be generated by the system based on another user tag. For example, a key of a user tag may be the text “Home Weather” and a value of the user tag may be the weather information retrieved by the system based on a value of another user tag with a key “Home Address.”

At step 604, the user may configure settings for notification and monitoring parameters. In some embodiments, the user may configure settings for notification and monitoring parameters by selecting from a set of settings for notification and monitoring parameters configured by the administrator. In some embodiments, the settings for notification and monitoring parameters may include the query settings 113 and the alert settings 114. The query settings may include, for example, a query schedule and the alert setting may include at least an alert criterion and an alert procedure that corresponds to each alert criterion, as disclosed above.

As previously discussed, the alert criterion may define a condition in which the alert procedure is executed. In some embodiments, the alert criterion may be defined in terms of the response data provided by the user for a particular query. For example, the alert criterion may be defined such that the alert procedure is executed when a response data to a query is above or below a predetermined value. In another example, the alert criterion may be defined such that the alert procedure is executed when a response data for a query is above or below the cumulative average of the response data.

In some embodiments, the set of actions defined in the alert procedure may include contacting one or more people based on information user entered while configuring settings for notification and monitoring parameters or based on one or more user tags. For example, when an alert procedure is executed, system 100 may retrieve the value associated with the user tag with the key “Best Friend” and contact the user's best friend using the value of the user tag. The user tag value may be, for example, an email or a phone number, and system may determine the appropriate method of contacting the user's best friend depending on the type of contact information contained in the user tag.

In some embodiments, the set of actions defined by an alert procedure may include notifying a device associated with the administrator or third party, such as device 150. The notification may include the details of the response data and the query that caused the alert procedure to be executed. In some embodiments, the notification to the administrator or third party may include options for the administrator or third party to select. These options may include, for example, an option to contact one or more people (e.g., people the user has preauthorized or other healthcare professionals). In another example, these options may include an option to generate/load and send additional queries to the user.

In some embodiments, the user may determine whether they want notifications to be sent to their mobile devices, such as device 120. The notifications may be SMS notifications, for example.

At step 606, the account may be approved by an administrator. Subsequently, queries are sent to the user based on the query settings, and the user provides response data corresponding to each query to the system. Alternatively, a query may be, for example, an alert or a reminder that requires no response data from the user. In the next step, user's response data are analyzed, for example, by analytics module 115.

After the account is approved by an administrator, in some embodiments, at least one query may be sent to the user when an input data is received at server 110 from the user's device. In these embodiments, the input data may include a text data or a keyword that corresponds to one or more predetermined queries, and upon receiving the input data, server 110 may send the corresponding predetermined one or more queries to the user's device.

In some embodiments, server 110 of the system may generate and send at least one additional query to the user's device based on the analysis of the response data. Alternatively, server 110 of the system may load and send a set of preconfigured queries. Consider an example where the queries are directed to determining the user's level of depression. If the analysis of the response data from these queries indicate that the user may be depressed, a set of queries that has been validated and/or approved by FDA or similar authority for health interactions to diagnose depression may be loaded and sent to the user's device. In some embodiments, these additional queries may be automatically loaded and sent by the system 100 based on the analysis of the response data. In other embodiments, the administrator's input may be solicited, as discussed above. For example, a notification may be sent to the administrator based on the response data, and the notification may include options to generate/load and send additional queries to the user. In some examples, the administrator may load the additional queries to send from a library of query sets included in the system 100, for example in database 111. In these examples, each query set may be preconfigured based on a study and/or may be a validated set of queries for a certain condition.

In some embodiments, a clinical interface may be displayed to a device associated with the administrator during process 600. A “clinical interface” may be a graphical interface for providing filtered, concise data to an administrator in an intuitive and easy to comprehend format to facilitate the use of those compiled data in treatment planning and execution. Specifically, the administrator may determine the parameters, rules, and variables to measure for one patient, some patients or the entire population, and the clinical interface may provide a “snap shot” of how an individual patient is faring compared to the population as a whole. Thus, the administrator may use the clinical interface to determine how that patient would do in the future, under the same parameters.

In some embodiments, the clinical interface may display data to aid an administrator in determining a treatment. The clinical interface may also display summarized data with an option for the administrator to access complete data. In some embodiments, the clinical interface may be updated in real-time (e.g., the clinical interface may be updated when an additional response data from a user is received).

The clinical interface may display data that is charted in both raw and calculated formats. When data is analyzed for changes over a period of time, for example, each response data may be compared to the running mean of that response data. This process may serve to make all observations relative to the individual rather than an external standard or value.

In some embodiments, the clinical interface may include a table view. In one example, the table view may display a value representing a change in the response data over a period of time. In another example, the table view may display a value representing a cumulative mean of the response data. In yet another example, the table view may display a value calculated based on the response data received from multiple patients in the same treatment group. In this example, the clinical interface may aid the administrator in evaluating how each patient's response data compares to that of their treatment group. The same treatment group may be defined as, for example, a group of patients participating in the same treatment sequence and/or at the same facility.

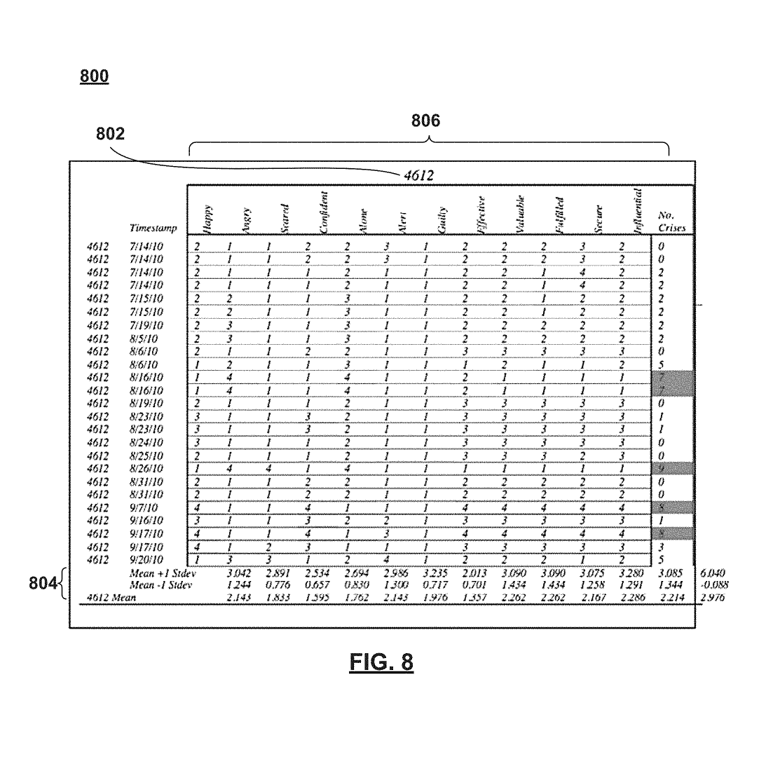

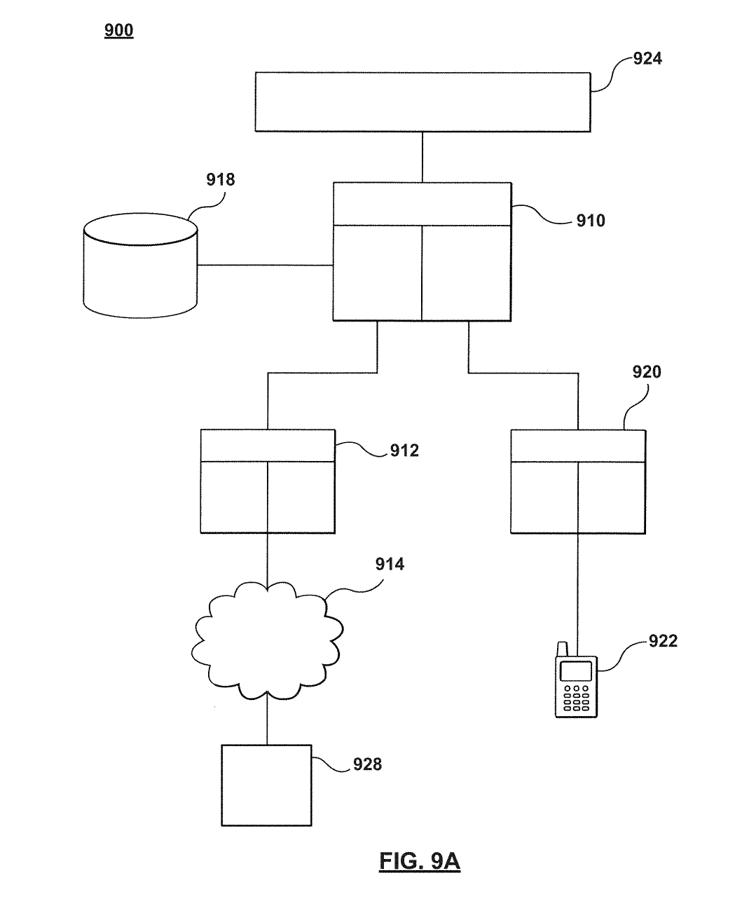

FIG. 8 shows an example clinical interface 800, consistent with disclosed embodiments. Clinical interface 800 includes a table view 806 displaying response data by a patient and a label 802 identifying the patient. In some embodiments, clinical interface 800 may also include statistical data 804 which includes cumulative mean and mean+/−standard deviation for each column in the table view.

FIG. 6B is a flow diagram of an exemplary process 610 for setting up user information, consistent with disclosed embodiments. In some examples, process 610 may be performed at step 604 of FIG. 6A. At step 612, the user may use one or more input devices to provide information requested by a system 100. In some embodiments, the requested information may include information to protect the user account. For example, the requested information may include a user's biometric data, digital signature, user identification and password, user security encoding information, or a combination thereof. In some embodiments, the requested information may include attribute parameters, which are elements that would allow the system to better assign questions/notifications worded in a manner most likely preferred by the user. Attribute parameters may also enable the system to group the users with similar attributes for chats, support, etc. The user thus establishes the notification scheme based on their own interactive lifestyle monitoring needs. In some embodiments, the requested information may include user's contact information, information for setting up mobile notification, and/or user's email address.

At step 614, the user may configure settings for notification and monitoring parameters. The queries can be set-up in a plurality of ways, for example, single query, multiple query with no immediate response required, or multiple query with a response dependent on input. The user may determine the nature of the one or more elements measured or may be pre-determined by a third party or administrator for the user. Alternatively, the administrator may provide a range of pre-configured queries from which the user may select from. In addition, a user may desire notifications for multiple events (e.g., body temperature, blood pressure, etc.). A user may also specify that the response can be sent via alternate methods than those used to send reminders (e.g., SMS notification is sent to a mobile device but the response is sent via a computer terminal). The parameters for analysis for triggering the notification (i.e. a reminder or an alert) can be established either by the user or by a third party administrator.

FIG. 6C is a flow diagram of an approval process 620, consistent with disclosed embodiments. Process 620 begins after the user submits all relevant and required information to a system administrator or manager (“administrator”) for approval. The submitted information may include the settings for notification and monitoring parameters. In some examples, process 620 may be performed at step 606 of FIG. 6A.

At step 622, the administrator receives the approval request that includes settings for notification and monitoring parameters.

At step 624, the administrator may approve or deny the request. In some embodiments, the administrator may modify the settings for notification and monitoring parameters prior to step 624. For example, the administrator may alter, add, or remove a query schedule, alert criterion, and alert procedure. In some embodiments, the administrator may also generate additional queries associated with the user account based on one or more user tags. In an example where a user tag has the text “delivery date” as the key and the user tag is associated with the user's account, the administrator may instruct the system 100 to generate additional query schedules for significant post-partum dates (e.g., 1 week, 1 month, and/or 3 months after the delivery date) based on the value of the user tag (i.e. date of delivery). In some embodiments, these additional query schedules may be manually created by the administrator. In some embodiments, the device used by the administrator may aid the generation of additional notifications. For example, the device used by the administrator may display one or more suggested queries schedules based on one or more user tags. Therefore, instead of creating the additional queries by manually entering required information, the administrator may create the additional queries by simply selecting one or more of the suggested notifications. The suggested notifications may be generated using one or more algorithms pre-programmed in the system 100, and these algorithms may be derived from one or more clinical studies or in consultation with a medical professional. Additionally, these suggested notifications may be generated at server 110 of the system 100, or alternatively, at a device such as device 120 or device 150.

After the approval process, approval information may be sent from the device used by the administrator to server 110 of system 100. The approval information may include information relating to whether monitoring scheme proposed by the user is approved, and may optionally include modified settings for notification and monitoring parameters.

Server 110 of system 100, upon receiving the approval information, may activate the proposed monitoring scheme (or the modified monitoring scheme, if included in the approval information).

Additionally, upon activation of a monitoring scheme, server 110 of system 100 may generate queries based on the query settings of the activated monitoring scheme and send the generated queries to the user's device based on the query schedule of the activated monitoring scheme.

In embodiments where one or more user tags are created and associated with the user account, notifications and queries may be generated based on one or more user tag values. In an example where a key of a user tag is the text “nick name,” the query may use the value of the user tag, which is the preferred nickname of the user, instead of the full name of the user. In another example, a key of a user tag may be the text “Capitals Fan” and the value of the user tag may be the latest, dynamically retrieved score of a hockey game involving the hockey team, Capitals. In this example, a notification may include the score of the hockey game at the time it is generated.

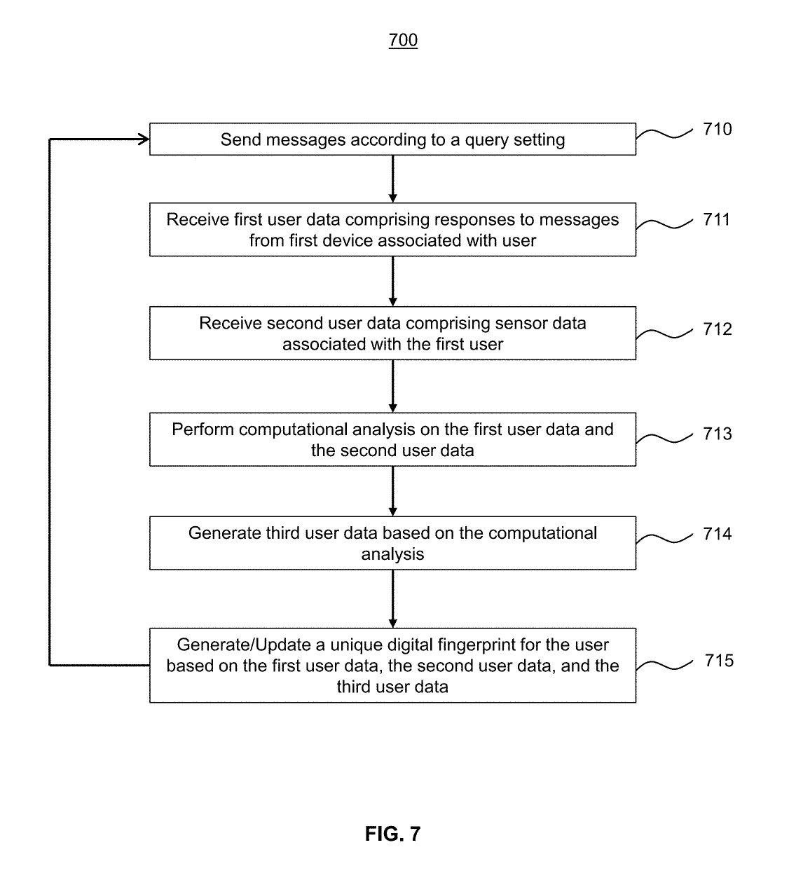

FIG. 7 illustrates a flowchart of an exemplary process 700 for monitoring patient health and generating a unique digital fingerprint for a patient, such as user 121. Process 700 starts at step 710. In step 710, server 110 sends messages to user device 120 associated with user 121. The messages may be generated based on information stored in database 111, user profile 112, and/or query settings 113. For example, user profile 112 may include information relating to the user's medical records, medical diagnoses, and/or specific medical information input by user 121 during registration or thereafter. Database 111 may include information regarding query templates and messaging protocols relating to the user's medical information stored in user profile 112. In some embodiments, database 111 and/or user profile 112 may include information gathered from a social media account of user 121 or from user 141. Query settings 113 may include parameters dictating the timing and frequency of the messages sent to user device 120, for example.

Server 110 sends the generated messages to device 120 over a communications network via network interface 116. The messages may include a visual or audio prompt for user 121 to provide responses. For example, a message may visually or audibly ask user 121 to rate their sleep from the previous night, their current mood, or their alertness. The prompt may include a scale or options for a range of responses. In step 711, server 110 receives first user data once user 121 provides the response. The first user data may comprise the responses to the messages sent in step 710, and may include subjective quantitative and/or qualitative data relating to user 121. The first user data may be stored in database 111 or user profile 112.

In step 712, server 110 may receive second user data from a sensor of user device 120 or from a second user device associated with the first user 121. In some embodiments, second user device is a sensor device 130 comprising one or more sensors. In some embodiments, a sensor of user device 120 may comprise an ambient sensor such as a microphone or a camera, or a physiological sensor embedded or connected to user device 120. The one or more sensors may generate objective quantitative and/or qualitative data relating to user 121. For example, the one or more sensors may generate data relating to the heart rate, pulse rate, electrocardiography (EKG or ECG), respiration rate, body temperature, skin temperature, blood pressure, glucose levels, activity levels, body position or orientation, location, sound, or sleep activity of user 121. In some embodiments, the sensor data may be received at server 110 directly from the sensor device 130. In some embodiments, sensor device 130 may transmit the sensor data to user device 120, and server 110 may receive the sensor data relayed from user device 120. The second user data may be stored in database 111 or in a data structure for user profile 112.

In steps 713 and 714, server 110 performs a computational analysis on the first user data and the second user data to generate third user data. In some embodiments, third user data may include new data that did not exist individually within the first or second user data, and may be the result of inputting first and/or second user data into a predetermined function or performing a statistical analysis on first and/or second user data. In other embodiments, third user data may include information extracted from first user data or second user data. In some embodiments, extracted data may be combined to result in new combinations of isolated and correlated data indicative of relationships between the first user data and second user data. In some embodiments, analytics module 115 may receive the stored first user data and second user data from database 111 or user profile 112, or alternatively directly from devices 120, 130, 140, and/or 150, and perform the computational analysis utilizing one or more algorithms. By analyzing the quantitative and qualitative patient information responses and data, the analytics module 115 may generate a unique digital fingerprint for the user.

In some embodiments, the computational analysis performed by analytics module 115 includes analyzing the first user data received in response to queries sent to user device 120. For example, the analytics module 115 may compare the user's responses to data relating to the set of queries stored in database 111 to determine whether additional queries should be sent to user device 120. In addition, based on the user's responses, the analytics module 115 may determine that further queries should be sent to second user 141 via user device 140 to solicit further responses. The responses from second user 141 may then be compared to the responses from user 121 to determine if further action is needed.

In some embodiments, the computational analysis performed by analytics module 115 may include analyzing the second user data received from sensor device 130. Second user data may include data generated by one or more sensors in sensor device 130. Analytics module 115 may compare user responses to queries with data received from sensor device 130 to determine a course of action or further queries to send the user. For example, as illustrated in FIGS. 2-4, analytics module may compare a user's response to how they slept the previous night with sensor data from sensor device 130 to determine if there are any discrepancies. For example, analytics module 115 may compare the user's response that the user slept “Lousy” the night before to sensor data showing that the user had 9 hours of sleep. Based on the discrepancy, the system may then generate follow-up queries based on the analysis.

In some embodiments, the computational analysis performed by analytics module 115 includes analyzing different parameters relating to the user's health and/or sensor data. For example, the parameters may include an amount of awake time in the middle of the night, an amount of deep (REM) sleep, a number of times awake, a mood/level of anxiety the next day, a mood/level of anxiety before going to sleep, the extent of nightmares (if any), an amount of sleep in general, a spouse's perspective on mood, anxiety and thoughts on restlessness (i.e. to the extent the spouse was aware of or witnessed restlessness in the middle of the night or not), and insights on any of the above from children, other family members, friends, co-workers, or other third parties. These parameters may be considered by the analytics module 115 either singularly or in combination.

In some embodiments, analytics module 115 generates communication patterns for a user based on the user's responses to queries. In addition, the user's answers to queries may then be compared to the user's communication patterns to determine further patterns, changes in patterns, to create predictive models for the user, or to generate a unique digital fingerprint for the user. In some embodiments, computational analysis may also include determining third user data. For example, third user data may include determining transactional data such as the difference in time between sending a query to user device 120 and receiving the response from the user. The difference in time may be calculated using time stamp information relating to the queries and responses. The time differential data may then be merged with other data sets and analytics, including first and second patient information, to generate a unique digital fingerprint for the user.

In some embodiments, third user data may be generated based on data relating to a general population. For example, third user data may be generated based on statistical data relating to a population. Statistical data may relate to base levels for a population with respect to various data points. For example, the statistical data may relate to base level health data for particular data sets of user populations. For example, third user data may also be generated based on statistical data relating to the user. For example, the user's statistical data may be used to determine patterns or a baseline for that user. The determined patterns or baseline can then be used for comparison with updated data sets relating to the user. In some embodiments, the statistical data is received from a third party database or from database 111.

In some embodiments, third user data may be generated based on data received from a third party, such as a doctor, psychologist, counselor, friend, family member, clinician, or the like. For example, a third party may provide further analysis after reviewing the user data or results of analyses from analytics module 115. In some embodiments, third user data may be generated based on a combination of data relating to a general population, data relating to the particular user, and/or data received from third parties. In some embodiments, third user data may be generated based on the parameters discussed above relating to the user's health and/or sensor data.

In some embodiments, the lack of responses from the user in response to the queries may be used as an input by analytics module 115. For example, system 100 may determine whether a predetermined amount of time for receiving the first user data in response to a sent message is exceeded. If the threshold is determined to have been exceeded, the system may use the lack of response as an input to update the user's patterns and/or unique digital fingerprint. In some embodiments, when the predetermined amount of time is exceeded and the system does not receive a response to the generated query, alert settings 114 may require that an alert or notification is sent to an authorized third party. For example, if the user does not submit a response to a query after thirty minutes, the system may check the alert settings 114 to determine if an alert should be sent. System 100 may then retrieve contact information for an authorized third party, such as third party 150, or a second user, such as user 141, and send a notification or alert to the third party or second user.

In step 715, server 110 may generate a unique digital fingerprint for the user. For example, analytics module 115 may use the analyzed data from steps 713 and 714 to generate a unique digital fingerprint for user 121 based on the first user data, the second user data, and/or the third user data. The unique digital fingerprint may be stored in a database, such as database 111. The unique digital fingerprint may be used by system 100 to update a treatment plan for the patient. For example, the unique digital fingerprint may be used by a third party to uniquely adapt medication and treatment to the user's unique digital fingerprint. A third party may be, for example, a hospital, doctor, psychologist, counselor, friend, family member, clinician, or the like The unique digital fingerprint may also be used by system 100 for predictive modeling of a patient. In some embodiments, the unique digital fingerprint for the user is constantly updated based on updated user data and analytics.

In some embodiments, system 100 may be used for monitoring patients to detect suicide ideation and pre-empt suicide attempts. For example, system 100 may monitor and analyze collected data to identify correlation between immediate periods, such as 10 to 14 days, of limited sleep and wide mood swings reflected in message responses, which may be an indicator for a suicidal event. In some embodiments, system 100 may send messages to a patient according to query settings. The patient may receive the messages on a user device, such as user device 120, soliciting responses from the patient relating to behavioral or subjective information. For example, the patient may receive messages soliciting feedback to the quality of the patient's sleep within the past day, or within a previous time period, such as a week. The system 100 receives the patient's response to the messages and compares the response to sleep data collected from a sensor device. Based on the comparison, system 100 may send further messages to the patient. For example, system 100 may send a message asking the patient to rate their mood for the day, based on a scale of 1 to 5, where 1 is great and 5 is lousy. System 100 may then receive the patient's response and make a further comparison to the previous responses and sleep data.

Based on the further comparison, system 100 may further solicit feedback from a user that is aware of the patient's circumstances. For example, system 100 may solicit feedback from a spouse of the patient. The spouse may receive a message requesting feedback to a similar query posed to the patient. For example, the spouse may be asked to rate the patient's mood. Based on the spouse's response, system 100 may then analyze the responses from the patient, the responses from the spouse, and the sleep data from the sensor(s) to determine patterns, changes in patterns, dissonance between the sensor data and the messaging responses, and predictive modeling for the patient. Based on the determination, system 100 may trigger an alert or suggest further action from the patient, such as an appointment to discuss the situation or check in with a health care worker. In some embodiments, non-responses from the patient are used to solicit further feedback or trigger alerts to a third party.

In some embodiments, the system may base the comparisons on thresholds or statistical baselines for a general population or the user's digital fingerprint. For example, the system may store or access data associated with one or more thresholds and patterns for detecting suicide ideation and attempts. For example, thresholds may be set correlating risk factors such as insomnia, hypersomnia, and/or nightmares with suicidal behavior. If the system determines the threshold is met, the system may solicit further feedback or trigger alerts to a third party. For example, W. Vaughn McCall, “The Correlation Between Sleep Disturbance and Suicide,” Psychiatric Times, Sep. 30, 2015, available at URL: <<http://www.psychiatrictimes.com/special-reports/correlation-between-sleep-disturbance-and-suicide>>, herein incorporated by reference in its entirety, discusses different statistics and correlations of parameters to suicide that may be used to develop the thresholds or statistical baselines for system 100.

In some embodiments, system 100 may be used for monitoring depression in patients. For example, system 100 may be used to detect depression in elderly or other patients. In this case, system 100 may send messages to check-in on the patient, based on a query schedule that is predetermined, set by the user/patient, or set by an administrator such as a caregiver. The check-in messages may solicit feedback relating to the patient's mood, activity level, and/or alertness. For example, the check-in messages may ask a patient “How do you feel today?” and request feedback in the form of a ranking of 1 to 5, where 1 is Great and 5 is Lousy. System 100 may also monitor and receive data from the patient, such as sleep or activity data relating to the patient from a sensor device. The received sensor data is compared to the patient's feedback to detect any changes in patterns. In some embodiments, the patient's non-responsiveness is used as a data point in the analysis. In other embodiments, the time different between the patient receiving a message and sending a response is used as a data point in the analysis. Based on the analysis, alerts can be sent to designated parties, such as a family member, clinician, or healthcare provider.

In some embodiments, system 100 may be used for monitoring first responders in active service, to predict the onset of stress or depression-related behavior conditions. For example, first responders may be sent continuous messages requesting feedback on the responder's situation and general disposition. Responses from the first responders may include location data so that the responders' constant movement is tracked. Comparing the first responder's responses and location data to sensor data, system 100 may provide analysis to facilitate triage, alerts, and/or directions during a critical or crisis situation. For example, sensor data relating to the first responder's physical state, including blood pressure, heart rate, etc. may be sent to system to facilitate the analysis. The analysis may be based on detected changes in patterns to a responder's responses and/or sensor data. In some embodiments, non-responses to messages may trigger alerts to third parties.

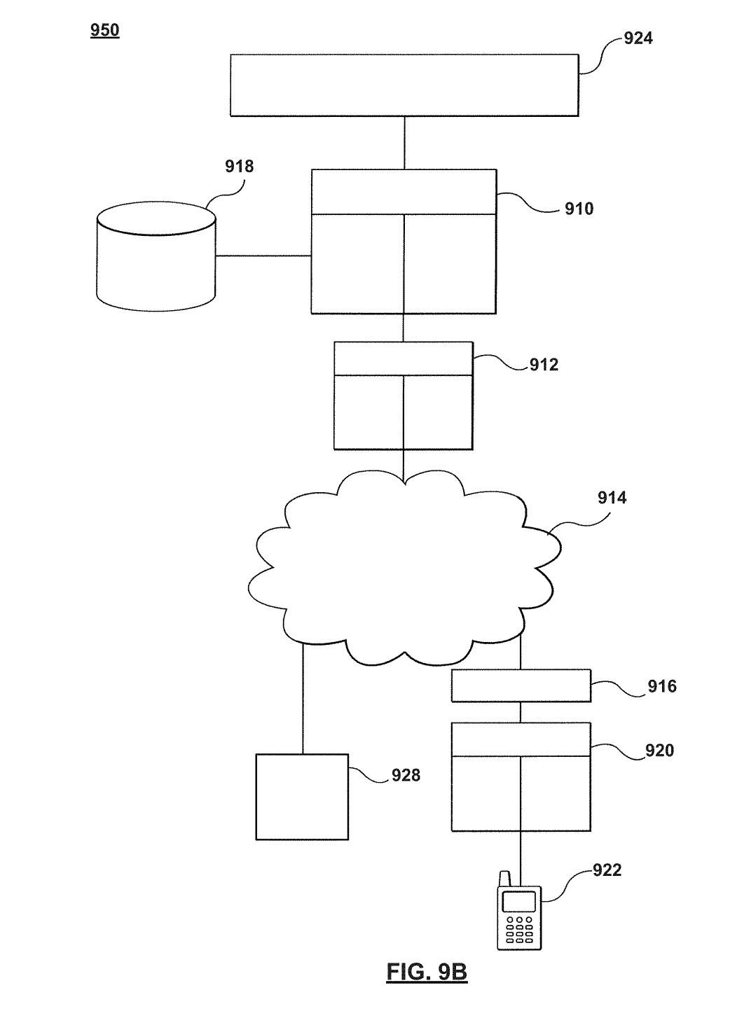

FIG. 9A illustrates an exemplary server system 900, consistent with disclosed embodiments. As shown in FIG. 9A, system 900 may include a server 910. Server 910 may further include a web server 912, a telephony server 920, a server application 924, and/or database 918. In some embodiments, server 910 may be connected to one or more devices or computers that implement the function of web server 912, a telephony server 920, a server application 924, and/or database 918. Database 918 receives and stores data associated with one or more processes disclosed herein, under the control of server 910 and/or one or more other processors in communication with database 918. In some embodiments, database 918 may store one or more programs or other computer-executable code that, when executed, cause one or more processors to perform functions and methods associated with disclosed embodiments. In some embodiments, database 918 may be integrated with server 910.

Still referring to FIG. 9A, system 900 may further include client devices such as a device 928 and/or a mobile device 922. In some embodiments, device 928 may include a computer connected to web server 912 through the a communications network 914, such as the Internet. Mobile device 922 may be connected to telephony server 920. In some embodiments, system 900 may include only one type of client device.

Still referring to FIG. 9A, server 910 includes server application 924. In some embodiments, server application 924 may include one or more software (e.g., a script or a program) that enables the processing steps described above and supports the described functions.

FIG. 9B illustrates another exemplary system 950, consistent with disclosed embodiments. System 950 is similar to system 900 of FIG. 9A except that telephony server 920 supports the connection between communication device 920 and server 910 via communications network 914 through a wireless gateway 916.

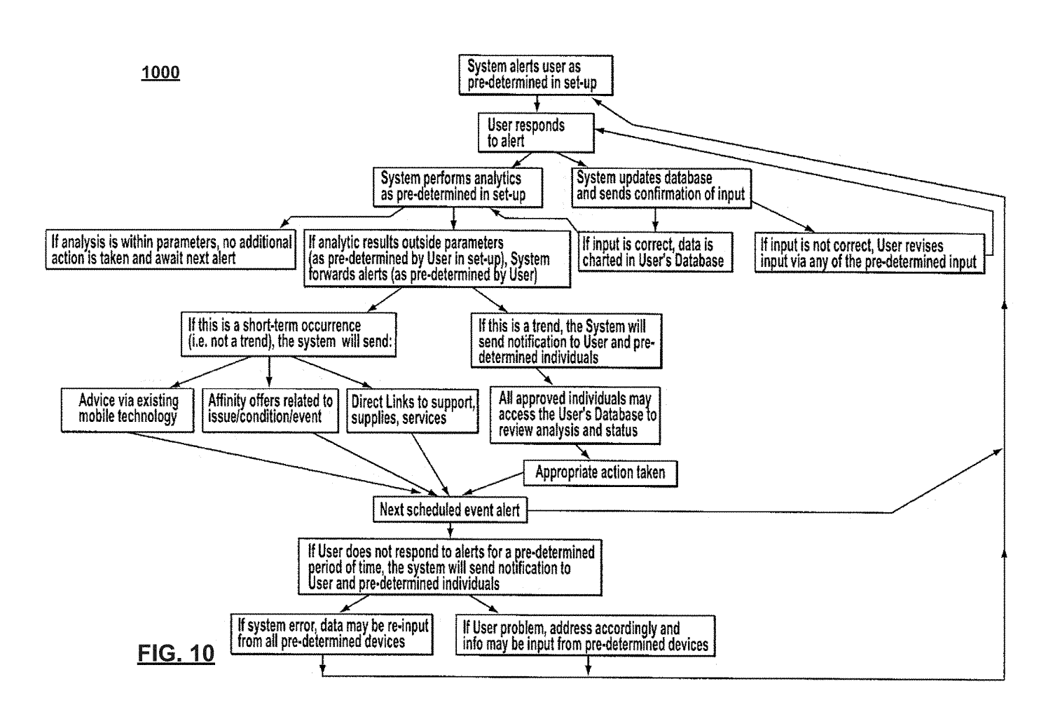

FIG. 10 illustrates a flow diagram of an exemplary process 1000 for the system workflow. An interface governs the relationship between the user and the system, including the responses provided by the user in response to a query or notification, and the analysis that the system performs.

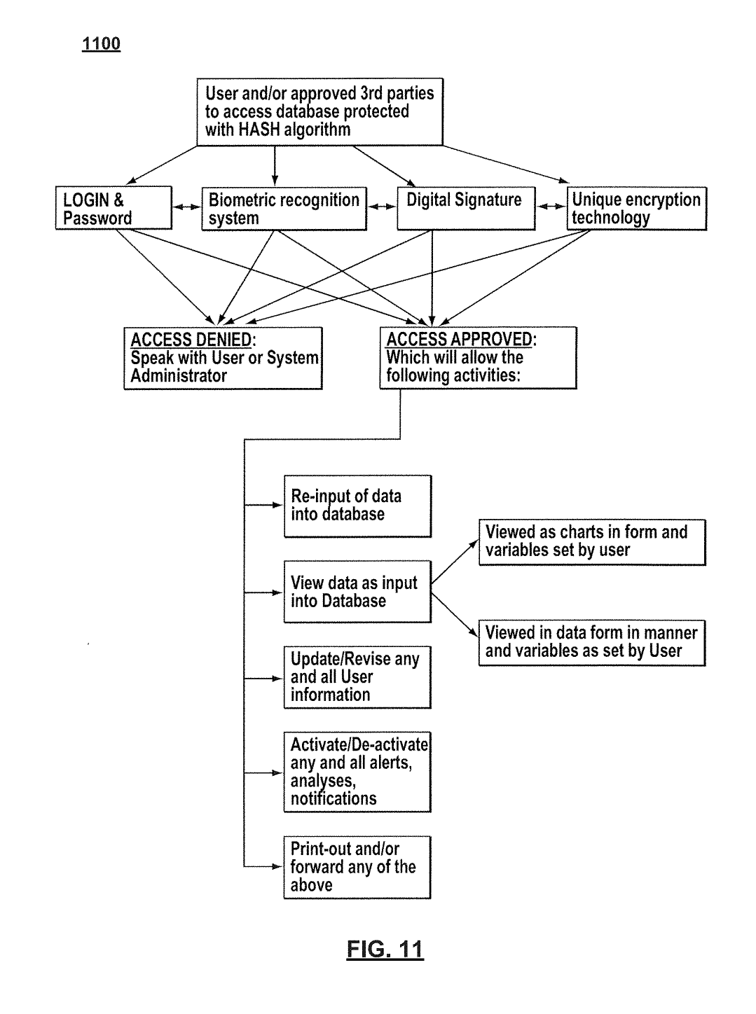

FIG. 11 illustrates a flow diagram of an exemplary process 1100 for protecting the information provided by the user. As illustrated in FIG. 11, access to database 111 may be protected by, for example, a HASH algorithm. This along with one or more other security techniques may ensure that the information remains secure.

Once the required parameters are specified, the user submits the info for approval. The administrator receives the request for approval. If the request is not approved, the administrator deletes the request and user cannot go any further. If approved, the user is sent a confirmation. The confirmation may be an email or SMS message, for example. Once the user replies, the system notifications scheme is established.

In an embodiment, once the notification scheme is established, the system provides notifications to the user. The user responds to a notification, and the system may determine that any response prior to the next notification pertains to the preceding notification. The system performs trend analysis based on parameters selected by the user. The system determines whether response is within or outside such parameters. If it is within parameters, the system sends a confirmation of the specific response. If outside the parameters, the system sends a confirmation of the specific response and provides additional notification that the user is outside its parameters. The system can also send notifications to a third party as indicated and approved by the user during application setup. The third party may access the user's database of information provided they have been given permission and the login and password information from the user.

In the event that the user does not respond for a specified period (with an example default setting being two days), the system will send a notification to the user notifying them of non-response and allowing them to review the account database to make required updates and/or to contact administrators/support.

In some embodiments, the user may access their database 111 or user profile 112 via a website using a login/password. Once the user has gained access, the user may have the following options: (i) non-wireless input, for example the user has the option at any time to input information directly into database via a computer with Internet access; (ii) charted information, for example viewing of their raw data in date order (as chosen by user) or for specific date period (as chosen by user), or user may view their data on an animated bar chart in date order (as chosen by user) or for specific period (as chosen by user); (iii) the user can update or revise information, including the information at initial set-up, e.g., SMS number, password or e-mail address; (iv) the user may activate or deactivate any notifications at any time; and (v) the user may print-out the database information from the web interface.

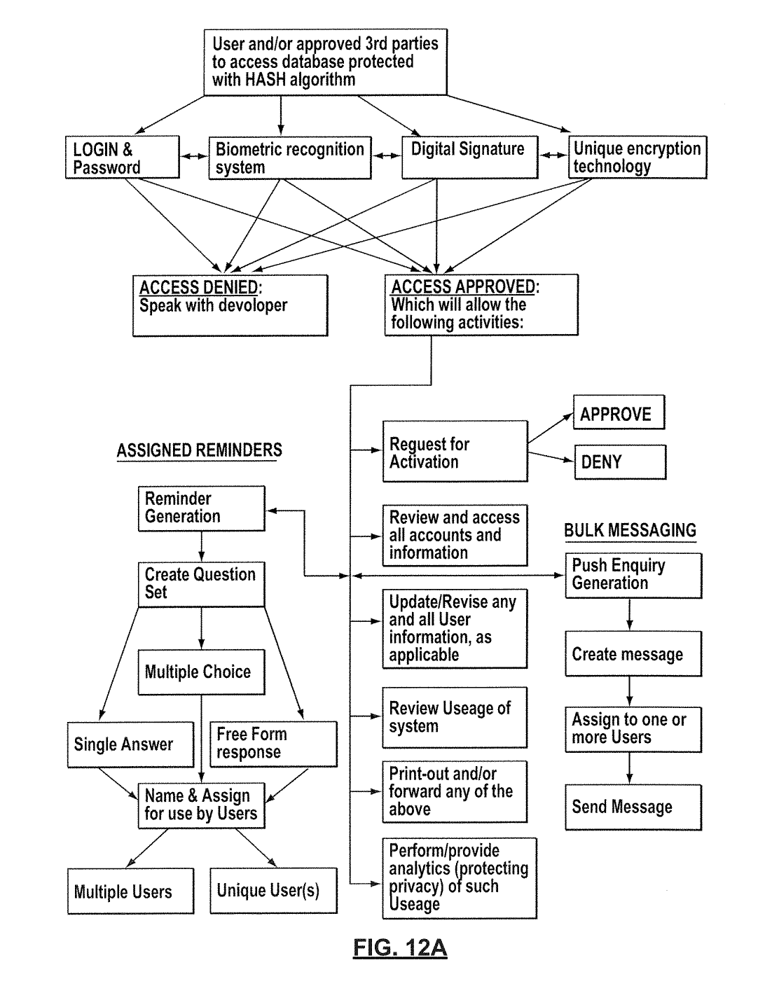

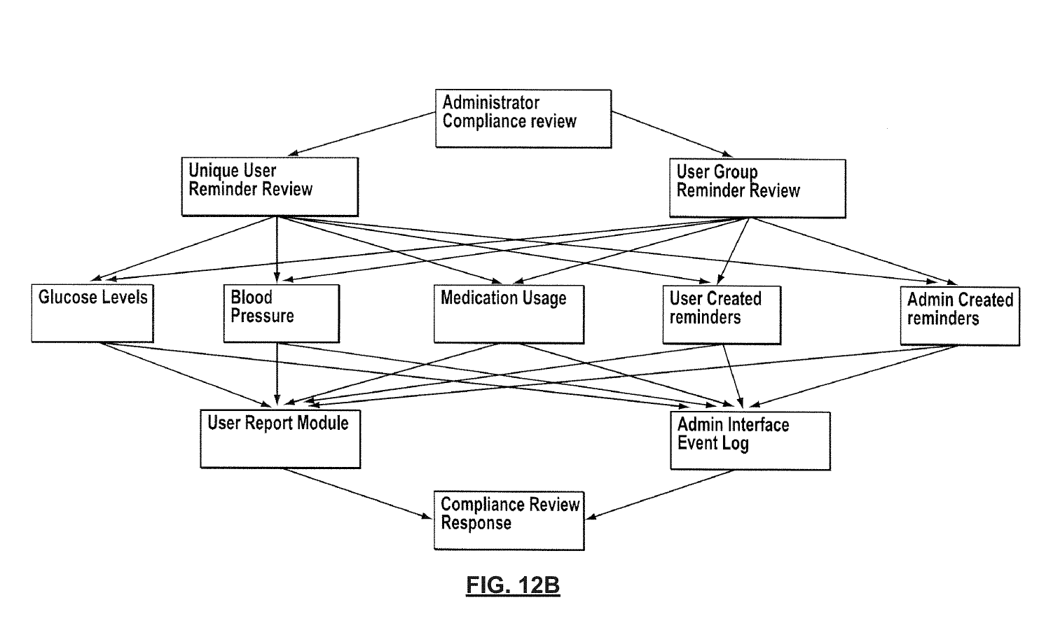

The administration interface of the disclosed system provides access to administrators to certain functions linked to the server 110. In an embodiment, these functions/resources are accessed via a series of web pages linked to a web server, such as web server 912. An administration interface may comprise a main page that prompts the user for login and password information. As an example, a flowchart illustrating a system administrator interface is provided in FIG. 12A. It should be understood that alternative methods for authentication are also contemplated by the disclosed system. These web pages, for example, enable administrators to approve all requests for activation, review and access all accounts (and respective databases), review usage of system via logs, etc., and provide an analysis of usage of system, accounts, etc. The administrator user compliance review is shown in FIG. 12B.

It should be understood that other functions/resources can be associated with the server and made accessible via selection from possible options via user commands described in the present disclosure.

The disclosed system also provides for a plurality of notification types. In one embodiment, the notifications are provided by the server to the user device using a messaging protocol. For example, the messaging protocol may comprise text messages including a single question or multiple questions. In a further aspect, notifications can be multiple questions wherein each subsequent question is dependent on the previous response. The disclosed system also contemplates multiple notification options, including audio alerts, including musical alerts, voice alerts, and/or simple tones or ringing.

These audio notifications can be in mp3 or other suitable formats. A visual alert can be provided where such visual notifications are supported by the user device. The notification can also be a direct alert linking to a third party. It should be understood that any notification or alert type mentioned herein can be implemented alone or in combination with other alert types, in accordance with the user's selections.

In some embodiments, musical notifications or alerts can be provided to the user device. In this regard, it is possible to correlate the particular musical piece to the various specific levels of notification or alert for the user. The parameter of these types of notifications can be set by the user and changed from time to time, in accordance with some embodiments.

In a further aspect of the disclosed system, the content of notifications can comprise specific information based on database analysis. For example, the notification content can be tailored (by the user) in relation to the database analysis relevant to, e.g., dietary requirements, exercise requirements, medical condition, individual reading or measurement, and/or support requirements.

In further aspects of the disclosed system, notifications can comprise further information and content for the user. For example, a notification can take the form of a video of a caregiver, or even of the user themselves, giving a quick lecture about the current status, corresponding to the notification level. The notification can also provide links to sites, phone numbers, and suppliers relevant to the level. Alternatively, the notification can directly provide a tie-in, via text, cell, e-mail, web, camera, etc., to the particular caregiver or support individuals, as customized by the user. The present disclosure contemplates each one of these possible notifications/alerts, and any combination thereof, with the type of notification and any corresponding trend analysis customizable by the user.

The information requested by each notification is dependent on the individual needs of the user. The disclosed system is capable of interactively monitoring a plurality of measurements. Tailoring the content of notifications may further improve the user's success at achieving adherence/compliance to a particular health regime.

Further, the disclosed system is not limited to quantitative information but is also operable to prompt the user for specific non-quantitative health or lifestyle related information related to any of the applications discussed herein. For example, the notification can consist of a simple question, such as “How do you feel?”, “Do you feel alert?”, and “Do you feel tired?”, etc. This in turn can result in additional questions to where these qualitative questions can further yield helpful information from the user and can be correlated to other data via the data analysis aspect of the disclosed embodiments. Such non-quantitative health or lifestyle related areas include dietary issues, mental health such as anxiety and exercise, to name a few.

Referring again to FIG. 1, server 110 may further include a processor 117 and a memory 118 coupled to the processor. In some embodiments, memory 118 stores instructions for the processor 117 to execute to implement functions and methods associated with disclosed embodiments. Memory 118 may also store a collection of program or database components such as an operating system 118a and graphical user interface (GUI) module 120. The operating system 118a may facilitate resource management and operation of the server 110. Examples of operating systems include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. GUI module 118b may process incoming and outgoing information between server 110 and devices 120, 130, 140, and/or 150.

Memory 118 may include, for example, RAM, ROM, or the like. Memory 118 may be operably connected to a database 111. Database 111 may connect to memory devices including, without limitation, memory drives, removable disc drives, or the like, employing connection protocols such as serial advanced technology attachment (SATA), integrated drive electronics (IDE), IEEE-1394, universal serial bus (USB), fiber channel, small computer systems interface (SCSI), or the like. The memory drives may further include a drum, magnetic disc drive, magneto-optical drive, optical drive, redundant array of independent discs (RAID), solid-state memory devices, solid-state drives, etc. Variations of memory 118 may be used for storing, for example, programs corresponding to the methods disclosed herein and/or outputs of the methods provided herein.

Server 110 may further include a central processing unit (“CPU” or “processor”) 117. Processor 117 may include at least one data processor for executing program components for executing user- or system-generated requests. A user may include a person, a person using a device such as those included in this disclosure, or such a device itself. The processor may include specialized processing units such as integrated system (bus) controllers, memory management control units, floating point units, graphics processing units, digital signal processing units, etc. The processor may include a microprocessor, such as AMD Athlon, Duron or Opteron, ARM's application, embedded or secure processors, IBM PowerPC, Intel's Core, Itanium, Xeon, Celeron or other line of processors, etc. Processor 117 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), or the like.

In some embodiments, execution of the sequences of instructions may be performed by a single computer system. In other embodiments, two or more computer systems may be coupled to perform the sequence of instructions in coordination with one another. The illustrated system may transmit and receive messages, data, and instructions, including program code (i.e., application code). Received program code may be executed by processor 117 as it is received, and/or stored in a disk drive, or other non-volatile storage for later execution. Additionally, in the same embodiments or other embodiments, the server, messaging and instructions transmitted or received may emanate from hardware, including operating system, and program code (i.e., application code) residing in a cloud implementation.

Further, it should be noted that one or more of the systems and methods provided herein may be suitable for cloud-based implementation. For example, in some embodiments, some or all of the data used in the disclosed methods may be sourced from or stored on any cloud computing platform.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A computerized system for monitoring patient health, the system comprising:
a storage device that stores a set of instructions; and
at least one processor configured to execute the stored instructions to perform operations including:
sending, to a client device associated with a patient,
messages according to a query setting;
receiving, from the client device associated with the patient, first patient data comprising responses to the messages;
receiving, from an electronic device associated with the patient and comprising a sensor, second patient data comprising sensor data corresponding to the patient;
performing computational analysis on the first patient data and the second patient data to generate third patient data; and
generating a unique digital fingerprint for the patient based on the first patient data, the second patient data, and the third patient data.

2. The system of claim 1, wherein performing computational analysis on the first patient data comprises comparing the responses to the messages to historical responses to previous messages for the patient.

3. The system of claim 1, wherein the messages and first patient data are time stamped, and performing computational analysis on the first patient data comprises computing a time difference between sending a message to the client device and receiving a response to the message from the client device.

4. The system of claim 1, wherein the electronic device associated with the patient comprises a wearable device.

5. The system of claim 4, wherein the sensor data comprises at least one of blood pressure, body temperature, heart rate, activity data, and sleep data.

6. The system of claim 1, the operations further comprising:
sending, to a computing device associated with an authorized person, messages according to the query setting; and
receiving, from the computing device, fourth patient data comprising responses to the messages;
wherein:
generating third patient data further includes performing computational analysis on the fourth patient data; and
generating the unique digital fingerprint for the patient is based on the first patient data, the second patient data, the third patient data, and the fourth patient data.

7. The system of claim 1, wherein the unique digital fingerprint is used to generate at least one of a predictive model or behavioral patterns for the patient.

8. The system of claim 1, the operations further comprising:
determining whether a predetermined amount of time for receiving the first patient data in response to a sent message is exceeded; and
sending an alert to an authorized third party based on the determination.

9. The system of claim 1, the operations further comprising:
receiving, from the client device associated with the patient, parameters for the query setting, the parameters comprising a query schedule;
receiving, from the client device associated with the patient, parameters for an alert setting, the parameters comprising an alert criterion and an alert procedure; and
generating messages according to the query setting.

10. The system of claim 9, the operations further comprising:
generating alerts based on the alert setting; and
sending the alerts to an authorized third party.

11. The system of claim 1, wherein the messages comprise automated, pre-programmed messages.

12. The system of claim 1, the operations further comprising:
updating a treatment plan for the patient based on the generated unique fingerprint.

13. A computer-implemented method for monitoring patient health, the method comprising the following operations performed by at least one processor:
sending, to a client device associated with a patient, messages according to a query setting;
receiving, from the client device associated with the patient, first patient data comprising responses to the messages;
receiving, from an electronic device associated with the patient and comprising a sensor, second patient data comprising sensor data corresponding to the patient;
performing computational analysis on the first patient data and the second patient data to generate third patient data; and
generating a unique digital fingerprint for the patient based on the first patient data, the second patient data, and the third patient data.

14. The method of claim 13, wherein performing computational analysis on the first patient data comprises comparing the responses to the messages to historical responses to previous messages for the patient.

15. The method of claim 13, wherein the messages and first patient data are time stamped, and performing computational analysis on the first patient data comprises computing a time difference between sending a message to the client device and receiving a response to the message from the client device.

16. The method of claim 13, further comprising the following operations performed by the processor:
sending, to a computing device associated with an authorized person, messages according to the query setting; and
receiving, from the computing device, fourth patient data comprising responses to the messages;
wherein:
generating third patient data further includes performing computational analysis on the fourth patient data; and
generating the unique digital fingerprint for the patient is based on the first patient data, the second patient data, the third patient data, and the fourth patient data.

17. The method of claim 13, further comprising the following operations performed by the processor:
determining whether a predetermined amount of time for receiving the first patient data in response to a sent message is exceeded; and
sending an alert to an authorized third party based on the determination.

18. The method of claim 13, further comprising the following operations performed by the processor:
receiving, from the client device associated with the patient, parameters for the query setting, the parameters comprising a query schedule;
receiving, from the client device associated with the patient, parameters for an alert setting, the parameters comprising an alert criterion and an alert procedure; and
generating messages according to the query setting.

19. The method of claim 18, further comprising the following operations performed by the processor:
generating alerts based on the alert setting; and
sending the alerts to an authorized third party.

20. A tangible, non-transitory computer-readable memory device that stores a set of instructions that, when executed by at least one processor, cause the at least one processor to perform operations comprising:
sending, to a client device associated with a patient, messages according to a query setting;
receiving, from the client device associated with the patient, first patient data comprising responses to the messages;
receiving, from an electronic device associated with the patient and comprising a sensor, second patient data comprising sensor data corresponding to the patient;
performing computational analysis on the first patient data and the second patient data to generate third patient data; and
generating a unique digital fingerprint for the patient based on the first patient data, the second patient data, and the third patient data.
